(19) United States
(12) Patent Application Publication
(10) Pub. No.: US 2018/0305389 A1
(43) Pub. Date: Oct. 25, 2018

(54) BRANCHED ALCOHOL-BASED SUGAR SURFACTANTS

(71) Applicant: Dow Global Technologies LLC, Midland, MI (US)

(72) Inventors: Troy E. Knight, Missouri City, TX (US); Nermeen W. Aboelella, Pearland, TX (US); Sayeed Abbas, Houston, TX (US); Aaron W. Sanders, Houston, TX (US)

(73) Assignee: Dow Global Technologies LLC, Midland, MI (US)

(21) Appl. No.: 15/763,507

(22) PCT Filed: Sep. 7, 2016
(86) PCT No.: PCT/US2016/050458

§ 371 Date: Mar. 27, 2018

Related U.S. Application Data
(60) Provisional application No. 62/233,464, filed on Sep. 28, 2015.

(51) Int. Cl. C07H 15/08; C07H 1/06

(52) U.S. Cl. CPC C07H 15/08; B01J 27/12; C07H 1/06

(57) ABSTRACT

Prepare a branched alcohol-based sugar surfactant by: (a) providing an ether alcohol and a fully acetylated sugar where the ether alcohol has the structure of Structure (I); (b) coupling the ether alcohol with the acetylated sugar in the presence of a Lewis acid catalyst to form a branched glucoside acetate; and (c) deprotecting the glucoside acetate by removing the acetate moieties and replacing them with hydrogen atoms in the presence of a base to form a surfactant having the structure (II).

### BACKGROUND OF THE INVENTION

### Field of the Invention

The present invention relates to alkyl glucoside surfactants.

### Introduction

Surfactants for chemical enhanced oil recovery (CEOR) should possess hydrophobes with long alkyl tails (typically C12-C24) that help solubilize in the oil phase and exhibit stronger interfacial interactions at elevated temperatures, along with light alkyl chain branching to prevent liquid crystalline phases. Sugar surfactants have better salt tolerance and decreased temperature sensitivity as compared to ethoxylate-based surfactants, which makes them desirable for use in CEOR applications.

The most common commercial route to making sugar surfactants, in particular alkyl glucosides, involves a process known as Fisher glycosylation. Fisher glycosylation involves the acid-catalyzed coupling of an alcohol (usually linear C4-C12) and glucose. A large excess of alcohol (usually approximately six mole excess) is used to avoid unwanted polysaccharide formation, but also adds cost to the process by having to remove the un-reacted alcohol under heat/vacuum. Removal of the high boiling C12-C24 hydrophobes (alcohols) would make the Fisher glycosylation route extremely difficult, and potentially cost prohibitive. In addition, bulky C12-C24 alcohols (OH group is buried within the large branched alkyl backbones) would make achieving a high yield of an alkyl glucoside very challenging.

It is desirable to identify a method for preparing sugar surfactants that offers enhanced yield results without requiring even four mole excess of alcohol. Even more desirable is such a method that is suitable for preparing branched sugar surfactants from branched alcohols, even secondary alcohols. Branching sterically hinders reactivity of the alcohols, making formation of a sugar surfactant more difficult. Nonetheless, branched sugar surfactants are particularly useful in CEOR applications because branching helps prevent gelling and liquid crystalline phases from forming.

### BRIEF SUMMARY OF THE INVENTION

The present invention provides a method for preparing sugar surfactants that offers enhanced yield results without requiring even three mole excess of alcohol. Even more, the present invention offers such a method that is suitable for preparing branched sugar surfactants from branched alcohols, even secondary alcohols.

The present invention is a result of surprisingly discovering that even if the alcohol is branched, or even if it is a secondary alcohol, the presence of —CH₂CH₂O— moieties just prior to the terminal hydroxyl of the alcohol increases yields of the target sugar surfactant.

Even more surprising was a discovery that —CH₂CH₂O— moieties just prior to the hydroxyl of the terminal hydroxyl of the alcohol results in isomeric selectivity favoring formation of the β-isomer of the alcohol-based sugar surfactant.

In a first aspect, the present invention is a process comprising: (a) providing an ether alcohol and a fully acetylated sugar, the ether alcohol having structure (I):

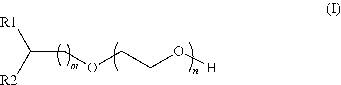

where R1 and R2 are independently selected from alkyl groups having from 4 to 16 carbon atoms, m is in a range of zero to ten and n is in a range from three to 40; (b) coupling the ether alcohol with the acetylated sugar in the presence of a Lewis acid catalyst to form a branched glucoside acetate; and (c) deprotecting the glucoside acetate by removing the acetate moieties and replacing them with hydrogen atoms in the presence of a base to form a surfactant having the structure (II):

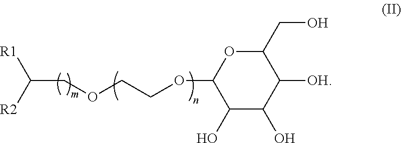

The process of the present invention is useful for preparing branched alcohol-based surfactants.

### DETAILED DESCRIPTION OF THE INVENTION

“And/or” means “and, or alternatively”. Ranges include endpoints unless otherwise stated.

Test methods refer to the most recent test method as of the priority date of this document unless a date is indicated with the test method number as a hyphenated two digit number. References to test methods contain both a reference to the testing society and the test method number. Test method organizations are referenced by one of the following abbreviations: ASTM refers to ASTM International (formerly known as American Society for Testing and Materials); EN refers to European Norm; DIN refers to Deutsches Institut für Normung; and ISO refers to International Organization for Standardization.

The present invention is a process for preparing a surfactant of structure (II):

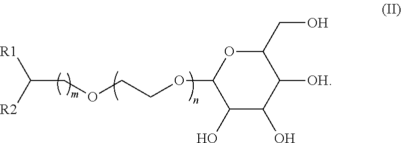

where R1 and R2 are independently selected from alkyl groups having from 4 to 16 carbon atoms, m is in a range of zero to ten and n is in a range from three to 40.

The process first requires providing an ether alcohol and a fully acetylated sugar. The ether alcohol has the structure of structure (I):

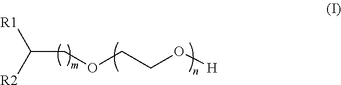

where R1 and R2 are independently selected from alkyl groups having four carbons or more, and can have five carbons or more, six carbons or more, seven carbons or more, eight carbons or more and even nine carbons or more while at the same time generally has 16 carbons or fewer, and can have 15 carbons or fewer, 12 carbons or fewer, 10 carbons or fewer, even nine carbons or fewer; m is selected from a value of zero or more and at the same time ten or less, typically three or less, more typically two or less and even more typically one or less; and n is selected from a value of three or more, typically five or more and can be seven or more, nine or more and even 11 or more while at the same time is 30 or less, typically 25 or less, more typically 20 or less, even more typically 15 or less and can be 14 or less, and even 13 or less. The ether alcohol has a particular characteristic of having —CH₂CH₂O— moieties just prior to terminal hydroxyl of the alcohol. The —CH₂CH₂O— moieties extend from a branched alkyl and extend directly off from a second degree position on the branched alkyl when m is zero.

The fully acetylated sugar has the structure of structure (III):

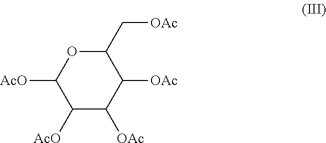 [SMILES: CC(=O)OCC1OC(OC(C)=O)C(OC(C)=O)C(OC(C)=O)C1OC(C)=O]

where Ac refers to an acetyl moiety.

The process of the present invention includes coupling the ether alcohol with the acetylated sugar in the presence of a Lewis acid catalyst to form a branched glucoside acetate. The coupling reaction is typically carried out in a solvent such as methylene chloride or chloroform. The concentration of ether alcohol is typically in a range of 1.2 to 2.7 molar. The concentration of acetylated sugar is typically 1.23 to 1.33 molar. Desirably, the molar ratio of ether alcohol to acetylated sugar is less than 3:1 and can be 2:1 or less while at the same time is 1:1 or more.

The Lewis acid catalyst can, in the broadest scope of the invention, be any Lewis acid. Particularly desirable Lewis acids for use in the coupling reaction include any one or any combination of more than one selected from a group consisting of boron trifluoride (for example, boron trifluoride gas, boron trifluoride diethyl etherate, boron trifluoride dimethyl etherate), stannic chloride, aluminum chloride, zinc trichloride and ferric chloride. However, one particularly desirable Lewis acid catalyst is boron trifluoride either as a gas or as a diethyl etherate. Generally, the concentration of Lewis acid catalyst in the coupling reaction is in a range of 1.1 to 2.7 molar.

Add a saturated aqueous solution of sodium bicarbonate to neutralize the Lewis acid catalyst once the coupling reaction has completed. Separate off the aqueous phase (for example, using a separatory funnel). Dry the organic phase using magnesium sulfate. Filter off the magnesium sulfate. Remove solvent, typically under reduced pressure, to yield the isolated branched glucoside acetate.

After forming and isolating the branched glucoside acetate, deprotect the glucoside acetate by removing the acetate moieties and replacing them with hydrogen atoms in the presence of base to form the surfactant of structure (II). The base is desirably a base selected from a group consisting of AMBERLITE™ resin beads and sodium methoxide. AMBERLITE is a trademark of Rohm and Haas Company. AMBERLITE resin beads are particularly desirable because it is easy to isolate form the reaction mixture once the reaction is complete. Generally, use approximately 20 grams of AMBERLITE resin beads in 30 milliliters methanol to treat the isolated branched glucoside acetate. Suitable AMBERLITE resin beads include AMBERLITE IRA 400(OH) resin beads.

The deprotection reaction is generally run in a solvent. Suitable solvents include low boiling alcohols such as any one or any combination of more than one selected from a group consisting of methanol, ethanol, propanol and butanol. Methanol is preferred as a solvent because it is most readily removed at the end of the reaction.

Generally, it is advantageous to conduct the deprotection reaction by dissolving the branched glucoside acetate into a solvent. Add the base at room temperature and allow to stir for 12 hours or more. Remove the base (for example, filter to separate AMBERLITE beads) and then evaporate off the solvent to yield the branched glucoside-based surfactant.

The deprotection reaction converts the branched glucoside acetate into a surfactant of structure (II) where R1, R2, m and n values for the ether alcohol and the resulting surfactant are the same.

The present process is capable of producing higher yields of acetylated alcohol-based sugars in the first step of the process and, hence, ultimately produce higher yield of alcohol-based sugar surfactant at the end of the process than similar processes where the ether alcohol starting material is free of —CH₂CH₂O— moieties just prior to the terminal hydroxyl of the alcohol. Likewise, the process of the present invention produces fewer by-products and produces higher yields at a given concentration of ether alcohol starting material than similar processes where the alcohol starting material is free of —CH₂CH₂O— moieties just prior to the terminal hydroxyl of the alcohol. This is particularly true for ether alcohols where the —CH₂CH₂O— moieties extend out from a second degree position on an alkyl relative to a similar secondary alcohol without the of —CH₂CH₂O— moieties.

It was also surprisingly discovered that the ether alcohol containing the —CH₂CH₂O— moieties just prior to the terminal hydroxyl of the alcohol produced more β-isomer of the glucoside acetate than a similar ether alcohol without the —CH₂CH₂O— moieties, indicating that the —CH₂CH₂O— moieties enhance isomeric selectivity in producing the glucoside acetate as well as the ultimate sugar surfactant.

### EXAMPLES

Sugar Surfactants from 2-Butyloctanol-Based Alcohols

2-butyloctanol has the structure of structure (I) where R1 is a six carbon alkyl, R2 is a four carbon alkyl, m is one and n is zero. One suitable commercially available 2-butyloctanol is available under the trade name ISOFOL™ 12. ISOFOL is a trademark of SASOL Germany GMBH.

2-butyloctanol-(EO)₆ is similar to 2-butyloctanol except there is an average of six —CH₂CH₂O— moieties just prior to the alcohol hydroxyl. Therefore, it has the structure of structure (I) where R1 is a six carbon alkyl, R2 is a four carbon alkyl, m is one and n is six. Prepare 2-butyloctanol-(EO)₆ in the following way.

Charge a nine-liter autoclave reactor with 553.7 grams (g) 2-butyloctanol and 4.48 g of 45 wt % aqueous potassium hydroxide at 100° C. with vacuum (70-100 millimeters mercury) for 2.5 hours. Measure the water by Karl Fisher titration (0.05% water). Heat the remaining catalyzed 2-butyloctanol initiator (525.4 g) with agitation to 135° C. and then meter in 744.5 g of ethylene oxide over three hours at 135° C. Once ethylene oxide feed is complete, continue agitating at 135° C. for seven hours to ensure ethylene oxide is consumed. Cool the reactor to 65° C. and drain the contents (1226.5 g). Neutralize the sample with magnesium silicate at 100° C. for one hour and then pull a vacuum to remove residual water. Allow the resulting slurry of magnesium silicate and product to cool and the filter to obtain the final product.

The acetylated sugar has the structure of structure (III) and is obtainable commercially from, for example, Sigma-Aldrich.

### Comparative Example A

Dissolve 7.56 g of β-D-glucose pentaacetate and 3.97 g of 2-butyloctanol in ten milliliters of dichloromethane. Add 3.02 grams of boron trifluoride diethyl etherate drop-wise over one minute and stir for 48 hours at 21° C. Add a saturated solution of sodium bicarbonate to the reaction mixture and shake the mixture. Transfer the two-phase mixture to a separatory funnel and remove the lower aqueous phase. Dry the organic phase over magnesium sulfate while stirring for 30 minutes. Filter the organic phase to separate out the magnesium sulfate and remove the dichloromethane under reduced pressure to yield the resulting branched glucoside acetate. Dissolve 10 g of the branched glucoside acetate into 30 milliliters of methanol. Add 20 grams of AMBERLITE IRA 400(OH) resin beads at 21° C. and stir for 12 hours. Filter the solution to remove the AMBERLITE resin beads and rinse the beads with methanol to isolate the methanol solution. Remove methanol from the isolated solution by rotary evaporation at 35° C. to obtain the branched glucoside-based surfactant (Comparative Example A).

Carbon-13 nuclear magnetic resonance (¹³C NMR) spectroscopy of the glucoside acetate obtained by coupling the ether alcohol with the acetylated sugar reveals a 65% yield of glucoside acetate with a significant amount of unreacted glucose pentaacetate and an unidentified by-product. Deprotection, even if 100% effective, can only result in a final yield of 65% branched alcohol-based sugar surfactant.

### Example 1

### Prepare Example 1 in a Similar Manner as Comparative Example A Except Use 2-butyloctanol-(EO)₆ Instead of 2-butyloctanol

Dissolve 4.84 g of β-D-glucose pentaacetate and 6.49 g of 2-butyloctanol-(EO)₆ in ten milliliters of dichloromethane. Add 1.94 grams of boron trifluoride diethyl etherate drop-wise over one minute and stir for 48 hours at 21° C. Add a saturated solution of sodium bicarbonate to the reaction mixture and shake the mixture. Transfer the two-phase mixture to a separatory funnel and remove the lower aqueous phase. Dry the organic phase over magnesium sulfate while stirring for 30 minutes. Filter the organic phase to separate out the magnesium sulfate and remove the dichloromethane under reduced pressure to yield the resulting branched glucoside acetate. Dissolve 10 g of the branched glucoside acetate into 30 milliliters of methanol. Add 20 grams of AMBERLITE IRA 400(OH) resin beads at 21° C. and stir for 12 hours. Filter the solution to remove the AMBERLITE resin beads and rinse the beads with methanol to isolate the methanol solution. Remove methanol from the isolated solution by rotary evaporation at 35° C. to obtain the branched glucoside-based surfactant (Example 1).

Carbon-13 nuclear magnetic resonance (¹³C NMR) spectroscopy of the glucoside acetate obtained by coupling the ether alcohol with the acetylated sugar reveals a 78% yield of glucoside acetate with a greater conversion of glucose pentaacetate. Moreover, it is evident that a only the β-isomer of the glucoside acetate (101 ppm shift in the NMR) is observed and no α-isomer at 96 ppm shift is evident, indicating that the ether alcohol containing the —CH₂CH₂O— moieties achieves greater isomeric selectivity than the reaction with a similar ether alcohol without the —CH₂CH₂O— moieties.

Deprotection of the glucoside acetate is expected to go to completion and produce an overall 78% yield of the branched alcohol-based sugar surfactant (Example 1) having the structure of structure (II) where R1 is a six carbon alkyl, R2 is a four carbon alkyl, m is one and n is six.

Example 1 illustrates the surprising effect of both achieving high (in this case, 78%) overall yield of branched alcohol-based sugar surfactant and high β-isomer selectivity when using a —CH₂CH₂O— moiety containing ether alcohol starting material.

Sugar Surfactants from Secondary Alcohol Ethoxylate

### Example 2

Prepare a branched alcohol-based sugar surfactant in a similar manner as Comparative Example A except use a secondary alcohol ethoxylate instead of the 2-butyloctanol. The secondary alcohol ethoxylate is a mixture of secondary alcohol ethoxylates of Structure (I) where R1 is a four carbon alkyl, R2 is a six to ten carbon alkyl, m is zero and n is five. This secondary alcohol ethoxylate is commercially available as TERGITOL™ 15-S-5. TERGITOL is a trademark of Union Carbide Corporation.

Dissolve 5.21 g of β-D-glucose pentaacetate and 6.16 g of the secondary alcohol ethoxylate in ten milliliters of dichloromethane. Add 2.08 grams of boron trifluoride diethyl etherate drop-wise over one minute and stir for 48 hours at 21° C. Add a saturated solution of sodium bicarbonate to the reaction mixture and shake the mixture. Transfer the two-phase mixture to a separatory funnel and remove the lower aqueous phase. Dry the organic phase over magnesium sulfate while stirring for 30 minutes. Filter the organic phase to separate out the magnesium sulfate and remove the dichloromethane under reduced pressure to yield the resulting branched glucoside acetate. Dissolve 10 g of the branched glucoside acetate into 30 milliliters of methanol. Add 20 grams of AMBERLITE IRA 400(OH) resin beads at 21° C. and stir for 12 hours. Filter the solution to remove the AMBERLITE resin beads and rinse the beads with methanol to isolate the methanol solution. Remove methanol from the isolated solution by rotary evaporation at 35° C. to obtain the branched glucoside-based surfactant (Example 2).

¹³C NMR spectroscopy of the glucoside acetate obtained by coupling the ether alcohol with the acetylated sugar reveals an 82% yield of glucoside acetate without a significant amount of unreacted glucose pentaacetate. The only isomer of the glucoside acetate observable in the ¹³C NMR spectrum is the β-isomer at 101 ppm shift; no α-isomer at 96 ppm shift was observable. Deprotection of the glucoside acetate is expected to go to completion, thereby resulting in an overall 82% yield of the β-isomer of the corresponding branched alcohol-based sugar surfactant (Example 2) having the structure of structure (II) where R1 is a four carbon alkyl, R2 ranges from six to ten carbon alkyl, m is zero and n is five.

Example 3 illustrates the surprising effect of both achieving high (in this case, exclusive) β-isomer selectivity but also an 82% yield of branched alcohol-based sugar surfactant when using a —CH₂CH₂O— moiety containing ether alcohol starting material.

### Example 3

Prepare a branched alcohol-based sugar surfactant in a similar manner as Example 2 except use 2.0 molar equivalents of secondary alcohol ethoxylate instead of 1.1 relative to acetylated sugar.

Dissolve 5.20 g of β-D-glucose pentaacetate and 11.20 g of the secondary alcohol ethoxylate in ten milliliters of dichloromethane. Add 3.78 grams of boron trifluoride diethyl etherate drop-wise over one minute and stir for 48 hours at 21° C. Add a saturated solution of sodium bicarbonate to the reaction mixture and shake the mixture. Transfer the two-phase mixture to a separatory funnel and remove the lower aqueous phase. Dry the organic phase over magnesium sulfate while stirring for 30 minutes. Filter the organic phase to separate out the magnesium sulfate and remove the dichloromethane under reduced pressure to yield the resulting branched glucoside acetate. Dissolve 10 g of the branched glucoside acetate into 30 milliliters of methanol. Add 20 grams of AMBERLITE IRA 400(OH) resin beads at 21° C. and stir for 12 hours. Filter the solution to remove the AMBERLITE resin beads and rinse the beads with methanol to isolate the methanol solution. Remove methanol from the isolated solution by rotary evaporation at 35° C. to obtain the branched glucoside-based surfactant (Example 3).

¹³C NMR spectroscopy of the glucoside acetate obtained by coupling the ether alcohol with the acetylates sugar reveals a 100% yield of glucoside acetate. The only isomer of the glucoside acetate observable in the ¹³C NMR spectrum is the β-isomer at 101 ppm shift; no α-isomer at 96 ppm shift was observable. Deprotection of the glucoside acetate is expected to go to completion and thereby produce an overall 100% yield of the β-isomer of the corresponding branched alcohol-based sugar surfactant (Example 3) having the structure of structure (II) where R1 is a four carbon alkyl, R2 ranges from a six to ten carbon alkyl, m is zero and n is five.

Example 3 illustrates the surprising effect of both achieving high (in this case, exclusive) β-isomer selectivity but also a 100% yield of branched alcohol-based sugar surfactant when using a —CH₂CH₂O— moiety containing ether alcohol starting material.

1. A process comprising:
(a) providing an ether alcohol and a fully acetylated sugar, the ether alcohol having structure (I): 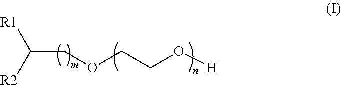
where R1 and R2 are independently selected from alkyl groups having from 4 to 16 carbon atoms, m is in a range of zero to ten and n is in a range from three to 40;
(b) coupling the ether alcohol with the acetylated sugar in the presence of a Lewis acid catalyst to form a branched glucoside acetate; and
(c) deprotecting the glucoside acetate by removing the acetate moieties and replacing them with hydrogen atoms in the presence of a base to form a surfactant having the structure (II):

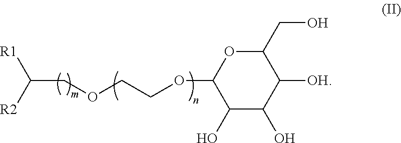

2. The process of claim 1, wherein the Lewis acid catalyst is boron trifluoride.

3. The process of claim 1, R1 is a linear 4 carbon alkyl and R2 is a linear alkyl with six to ten carbons.

4. The process of claim 1, wherein the base in step (c) is selected from a group consisting of AMBERLITE™ resin beads and sodium methoxide.

5. The process of claim 1, wherein n is in a range of 3 to 6, the Lewis acid catalyst is boron trifluoride and the base in step (c) is AMBERLITE™ resin beads.

6. The process of claim 1, wherein n is in a range of 5-6.

7. The process of claim 1, where the ether alcohol is present at a molar ratio of less than 3:1 and at the same time 1:1 or more relative to acetylated sugar in the coupling step (b).
